(19) United States
(12) Patent Application Publication
(10) Pub. No.: US 2006/0183227 A1
(43) Pub. Date: Aug. 17, 2006

(54) Method for gene transfer into the organelles of cells: direct gene transfer to mitochondria

(76) Inventor: Hong Bock Lee, San Diego, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 11/211,130

(22) Filed: Aug. 23, 2005

Related U.S. Application Data
(60) Provisional application No. 60/604,131, filed on Aug. 23, 2004.

(51) Int. Cl. C12N 15/867; C12N 15/86; C12N 5/08

(52) U.S. Cl. 435/456; 435/366

(57) ABSTRACT

Methods of gene transfer into cellular organelles are disclosed which provide the necessary conditions for genetic materials such as virus or recombinant DNA to be delivered directly into mitochondria from extracellular space in vitro or in vivo. Once inside the mitochondria, the genetic materials can then be translated according to the mitochondrial genetic code and mitochondrial versions of the proteins can be made. Other conditions and molecules can be added to enhance the delivery.

This application claims the benefit of priority of U.S. Provisional application Ser. No. 60/604,131, filed Aug. 23, 2004, the entire contents of which is incorporated herein by reference.

### FIELD OF THE INVENTION

The invention relates generally to a method for gene transfer into cellular organelles. More specifically the invention relates to the use of pressure and oxygenation to transfer nucleic acids into cellular organelles, particularly mitochondria.

### BACKGROUND OF THE INVENTION

Mitochondria are double layered membranous cellular organelles that act at the center stage of metabolism within the cell. According to the chemiosmosis theory, the intermembranous space of the mitochondria is defined by an outer membrane and an inner membrane and is important in ATP metabolism. The intermembranous space contains a hydrogen ion pool that is utilized by an ATP synthase to produce ATP. The inner membrane has an unusual phospholipid composition, which includes cardiolipin (diphosphatidylglycerol) and is particularly leak proof to small ions. This creates a pH gradient and a membrane potential across the impermeable inner membrane. The combination of the pH gradient and the membrane potential are known as the proton motive force. Because the lipid bilayer of the inner membrane is only about 5 nm thick, it is subject to enormous electrical forces. For example, an electric force as great as 150×10⁻³ volts across 5×10⁻⁹ meters may be produced, which results in 30,000,000 volts/meter (Scheffler I M, Mitochondria, Wiley-Liss, New York, 1999).

The mitochondrial double membrane acts as a size barrier. The outer membrane is known to be permeable to most small molecules of less than 5 kDa, while the inner membrane has an extremely restrictive size exclusion, as discussed above. However, when the inner membrane goes through a mitochondrial permeability transition, it allows molecules less than 1.5 kDa to pass. Molecules that are involved in metabolism are typically transported by a specialized protein or protein assembly, such as the translocase of the outer membrane (TOM) and the translocase of the inner membrane (TIM). (Scheffler I M, Mitochondria, Wiley-Liss, New York, 1999)

In the past, a number of publications reported that virus-like particles or viral proteins could be found within mitochondria based on morphologic observation and biochemical studies. As a result, it was believed that consensus viral proteins could be imported into mitochondria. More recently, the existence of virus or the viral genome in mitochondria has been cast in doubt. Because of the electric and size barrier of the double membrane of the mitochondria, it is widely believed that viral genetic material can not pass into mitochondria through the membranes. At present, only small fragments of RNA, mostly associated with an enzyme have been reported to enter the mitochondria (Entelis N S, et al. J Biol Chem 2001;276:45642-45653).

### SUMMARY OF THE INVENTION

A method of nucleic acid transfer into cellular organelles, such as mitochondria, is disclosed. In one embodiment, viral or recombinant genetic materials are delivered into mitochondria using pressure, oxygenation or both pressure and oxygenation. Once inside the mitochondria, the nucleic acid can then be translated according to the mitochondrial genetic code and mitochondrial proteins can be made. Other conditions and molecules can be utilized to enhance the delivery of nucleic acids into organelles.

One embodiment is a method for the introduction of nucleic acid into an organelle of a host cell and/or tissue, by contacting the host cell and/or tissue with the nucleic acid at a pressure of between about 1.0 and about 3.0 atmospheres for a time sufficient to introduce the nucleic acid into the organelle. In one aspect, an oxygenation of between about 50% and 95% is also applied. In a further aspect, the oxygenation is for a time of less than about 3 hours. In one embodiment, the method also includes a step of testing the cells for the presence of the nucleic acid within the organelle. In one aspect, the nucleic acid is transferred into the organelle through a direct route from the extracellular space without entering the cytoplasm. The organelle may be a mitochondrion or a chloroplast. In one aspect, the pressure is between about 1.0 and 2.5 atmospheres. Alternatively, the pressure is between about 1.6 and about 1.8 atmospheres. In one aspect, the oxygenation is between about 75% and 95%, preferably about 95%. In one aspect, the cell is a human tissue culture cell and/or the nucleic acid is viral nucleic acid, which may be carried by the virus. In one aspect, the nucleic acid is a vector.

In a further embodiment, the method involves contacting the cell with a protein or inorganic substance to enhance transfer of the nucleic acid and the protein may be a viral protein. In a further aspect, the nucleic acid is selected from the group of: viral nucleic acid, recombinant DNA, recombinant RNA, and mixtures thereof. In one aspect, the organelle is a mitochondrion and the nucleic acid is translated according to the mitochondrial genetic code. In a further aspect, the host cell is a non-natural host tissue or non-natural host cell. In a further aspect, the nucleic acid is modified by point mutagenesis to match the mitochondrial genetic code.

### DETAILED DESCRIPTION OF THE PREFERRED EMBODIMENT

A method of nucleic acid or gene transfer into cellular organelles, such as mitochondria, is disclosed. In one embodiment, viral or recombinant genetic materials are delivered into mitochondria using pressure, oxygenation or a combination of pressure and oxygenation. Once inside the mitochondria, DNA may be transcribed using the mitochondrial transcription machinery to produce mitochondrial RNA. RNA may be translated using the mitochondrial translation machinery to produce mitochondrial proteins based upon mitochondrial genetic code. In addition to pressure and oxygenation, other conditions can be modified and additional molecules can be used to enhance the delivery of nucleic acids into organelles.

Although the use of pressure and oxygenation are generally described in the alternative herein, a combination of pressure and oxygenation can also be used to deliver nucleic acid to the mitochondria.

Previous results (Paik K-H, U.S. Pat. No. 6,100,068, issued Aug. 8, 2000) showed the existence of viruses within mitochondria under experimental conditions, and surprisingly, in normatural host species. For example human hepatitis B virus was grown in rat hepatocytes and identified in the mitochondria of these cells, even though this virus naturally infects human hepatocytes. In the non-natural host cells, viral growth resembled natural infection in that the virus produced cccDNA and eAg within the mitochondria. This result is contrary to the conventional receptor theory in which it is held that a virus like HBV can only infect cells which express a specific viral receptor. Under normal conditions human HBV has never been known to enter rat or mouse hepatocytes. However, the results of these tests clearly showed that human virus entered the rat cells and the mitochondria in particular. One explanation for this finding is that the virus must have entered through an alternate pathway from the conventional receptor pathway. The ability of the virus to use the other pathway may have resulted from the specific experimental conditions. In the examples below, the different pathway was identified and methods were developed to utilize this newly identified pathway in molecular biology and particularly in the study of infectious disease.

A first insight into the pathway the virus was using came from applying a pressure of 2-3 atmospheres to the tissue or cells using the same growth and experimental conditions. The use of pressure seemed to trigger this alternate pathway. This type of hydrodynamic delivery of plasmid DNA or pressure mediated delivery of oligonucleotides has been previously reported (Liu F, et al. Gene Ther 1999;6:1258-1266; Mann M J, et al. U.S. Pat. No. 5,922,687) with regard to the passage of nucleic acids into the cytoplasm. However, Liu, et al. interpreted their data to indicate that rapid tail injection of DNA caused more than 40 mmHg of pressure on the hepatocytes which enabled DNA to pass through the plasma membrane en route to the nucleus. Mann, et al. later suggested that at 2 atmospheric pressure, 14mer or 18mer nucleotides entered endothelium or cardiac tissue and moved to the nucleus of these cells. However, in addition to these results, the previous results of Paik, et al. suggested that virus could be induced in this way to go through to the mitochondria.

A second hint at the alternate pathway came from electron microscopic observation. Occasionally virus-like particles were observed within a vesicle or tubule-like structure. This suggested that the virus-like particles were within a cellular organelle, such as the mitochondrion. Although it is possible that the virus-like particles entered by moving through the plasma membrane, cytoplasm and then through both mitochondrial membranes, this is highly doubtful, particularly because mitochondria do not possess the necessary viral receptors and because the mitochondrial membranes are highly impermeable even to very small molecules.

Thus, without being restricted to the following theory, it is believed that there is direct communication between the outside of the cell and the mitochondrion. Direct communication with the extracellular space suggests that the classical endosymbiosis theory may need to be modified. It suggests, in fact, that mitochondria retained the primitive trail for the entrance of molecules into a cell, but that this connection is kept closed under normal conditions allowing the mitochondrion to produce energy via the proton motive force. Again, without being restricted to a specific theory, high pressure is sufficient to open the normal connection or barrier between mitochondria and the extracellular space. Normally, this space should be sealed in order to keep the hydrogen gradient for ATP production intact. Typically viruses enter a cell by attaching to a cell surface receptor and either entering directly into the cytoplasm, being brought in within a vesicle or endosome, or injecting their nucleic acid into the cytoplasm. With respect to this new theory, it is possible that some viruses know how to open this channel to get into the mitochondria directly from the extracellular space of their natural host. Thus, for example, Hepatitis B virus produces eAg within the mitochondria and, thus, must enter into the mitochondria of human hepatocytes at some point in order to produce the viral protein eAg. Further, a recently described cell line SSP1 showed hepatitis C virus entered into mitochondria of human lymphocytes (Park S-S, U.S. Patent Application 60/583,945, filed Jun. 28, 2004, herein incorporated by reference in its entirety).

When taking this theory into account, it suggests that there are two entrances for a virus into a cell, either using the viral-specific receptor on plasma membrane, or using the direct channel between the extracellular space and the mitochondrion. However, it is known that a virus does not enter the cytoplasm of a non-natural host because the necessary receptor on the plasma membrane is not produced by the host. And in most cases, the virus is not capable of opening the direct channel between the extracellular space and mitochondria. Thus, the virus does not infect non-natural host cells. However, under a pressure of approximately 2-3 atmospheres, these channels appear to be opened artificially and to allow the virus to enter into the mitochondria of the non-natural host. Further, it is possible that some viruses do possess the ability to open this channel naturally, without the addition of outside pressure. In this case, the cells are natural hosts to these viruses, but may or may not possess the viral receptor.

Pressure has been used previously to allow delivery of a gene into the cytoplasm, allowing the passage of genetic material through the plasma membrane. However, the method and results described herein have surprisingly shown that a gene or nucleic acid can also be delivered directly to a cellular organelle, the mitochondrion, from the extracellular space. In Examples 1-7, this pathway was evaluated with a vector construct modified for mitochondrial expression.

It is of interest to localize nucleic acid to the mitochondria of cells because mitochondrially-expressed proteins may have differences in amino acid sequence and because it is likely that many parasitic proteins are naturally expressed in mitochondria. Most mitochondrial proteins are encoded by nuclear DNA that is transcribed and translated in the cytosol and then imported into the mitochondria. However, a certain percentage of the mitochondrial proteins are transcribed from mitochondrial DNA (mtDNA) and translated within the organelle itself using a non-universal genetic code. The mitochondrial system includes two ribosomal RNAs and 22 tRNAs. Comparison of the mitochondrial gene sequences with the amino acid sequences of the encoded proteins reveals that the genetic code within mitochondria is altered compared to the universal code used in the nucleus of eukaryotic cells and in most prokaryotes. For example, the UGA codon is a stop codon for protein synthesis in the universal code whereas UGA codes for a tryptophan in mitochondria, and the codons AGA and AGG code for arginine in the universal system but are stop codons in mammalian mitochondria. Because the mitochondria has its own transcription and translation systems, it is of interest to identify and produce proteins which are expressed in the mitochondria by parasites, as a function of a disease state, or naturally. Such proteins are likely to have different antigenic determinants and show a significant difference in size, shape and even function. In fact, some viral proteins may only be expressed in the mitochondria due to the presence of a codon which is read as a stop using the universal code, but becomes an amino acid when translated using the mitochondrial translation system. These mitochondrially expressed proteins may be used to produce vaccines, antibodies for diagnostics, and to identify therapeutics for use in treating such diseases.

Nucleic acid—The nucleic acid may be DNA, RNA, a mixture or any DNA or RNA-like substance known to one of skill in the art. The nucleic acid may be naked or comprise a carrier such as a vesicle, viral coat, or a vector. The nucleic acid may be inserted into an appropriate cloning vector using methods and vectors known to one of skill in the art. Possible vectors include plasmids or modified viruses. Of course the vector system may be chosen to be compatible with the host cell used. Such vectors include, but are not limited to, pHBVex (Paik K-H, 2000). The insertion into a cloning vector can, for example, be accomplished by ligating the DNA fragment of interest into a cloning vector which has complementary cohesive termini. Alternatively, the ends may be enzymatically or otherwise modified. Methods used for the production, purification and propagation of such vectors are known to one of skill in the art and may be, for example, identified in Sambrook, J., Fritsch, E. F., and Maniatis, T., in Molecular Cloning: A Laboratory Manual. Cold Spring Harbor Laboratory Press, NY, Vol. 1,2,3 (1989).

Gene—Most mitochondrial proteins are encoded by nuclear DNA that is transcribed in the nucleus and translated in the cytosol and then imported into the mitochondria. However, a certain percentage of the mitochondrial proteins are transcribed from mitochondrial DNA (mtDNA) and translated within the organelle itself using a non-universal genetic code. The mitochondrial system includes two ribosomal RNAs and 22 tRNAs. Comparison of the mitochondrial gene sequences with the amino acid sequences of the encoded proteins reveals that the genetic code within mitochondria is altered compared to the universal code used in the nucleus of eukaryotic cells and in most prokaryotes. For example, the UGA codon is a stop codon for protein synthesis in the universal code whereas UGA codes for a tryptophan in mitochondria, and the codons AGA and AGG code for arginine in the universal system but are stop codons in mammalian mitochondria. Thus, the nucleic acid may encode any protein which is to be translated using the genetic code specific to that organelle, for example, the mitochondrial genetic code. Genes of interest may be altered by point mutagenesis according to mitochondrial genetic code which are known to one of skill in the art (Sambrook et al, 1989).

Vectors and Control sequences—the nucleic acid and/or gene may be operably linked to a control sequence and may be incorporated into an expression vector for expression and a replicable vector for cloning and/or amplification. It is understood that many vectors can function for one or all of these uses. The control sequence can be any sequence which is used for the expression of an operably linked coding sequence in a particular host organism. The control sequences are typically suitable for eukaryotic cells, may contain promoters polyadenylation signals and enhancers, and may be specific to the specific organelle.

Pressure—The pressure need only be enough pressure to allow the delivery of nucleic acid into the mitochondrion of a typical animal cell, such as a tissue culture cell from an animal. The pressure used herein varied between about 1.0 and 3.2, preferably between about 1.6 and 3.0 atmospheres, including but not limited to: 1.6, 1.75, 1.8, 1.9, 2.0, 2.1, 2.2, 2.3, 2.4, 2.5, 2.6, 2.7, 2.8, 2.9 and 3.0. In a further embodiment, the pressure used is between about 1.6 and about 3.0 atmospheres. Preferably the pressure used is between about 1.6 and about 2.5 atmospheres. However, should the method be used for a plant cell or an atypical animal cell (one having a pellicle, cell wall, or comparable protective layer), a larger force might be needed or the removal of the protective barrier may be necessary.

The pressure and/or oxygenation may be applied at a constant rate, may oscillate, or may fluctuate. Further the pressure and oxygenation may be applied at the same time or in succession.

Oxygenation is the process of adding oxygen to the atmosphere in which the cells are held or grown. The amount of oxygenation is between about 50% and 95%, including but not limited to: 55%, 60%, 65%, 70%, 75%, 80%, 85%, 90%, and 92.5%. In one embodiment, the oxygenation used is 95%.

Temperature—The temperature is not envisioned to affect the process, thus the temperature is chosen to allow the cell the best growth advantage.

The culture conditions such as media, temperature and pH and the like can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Time—The time may be any amount of time necessary to move a sufficient amount of the nucleic acid into the organelle. The time may include but is not limited to less than three hours.

The invention can better be understood by way of the following examples which are representative of the preferred embodiments. The practice of the methods identified herein will employ, unless otherwise indicated, conventional techniques of molecular biology, microbiology, recombinant DNA and immunology, which can be reproduced by a person having an ordinary skill in the art. Exemplary methods may be used from references such as Ausubel et al., Current Protocols of Molecular Biology, Unit 3.16, John Wiley and Sons (1997) and Sambrook, J., Fritsch, E. F., and Maniatis, T., in Molecular Cloning: A Laboratory Manual. Cold Spring Harbor Laboratory Press, NY, Vol. 1,2,3 (1989).

### EXAMPLES

The following examples provide examples of methods of gene transfer directly into the mitochondria using pressure and/or oxygenation, methods to identify the optimum range of pressure, a method of gene transfer into a normatural host, and a method of establishing a transgenic mouse. The methods generally involve selecting a nucleic acid, mixing the nucleic acid with culture media, and increasing the pressure up to about 3.0 atmospheres for a period of time sufficient to transfer the nucleic acid into the organelle. The method may also involve applying oxygenation of about 95% for a period of time sufficient to transfer the nucleic acid into the organelle, which is typically less than 3 hours.

### Example 1

### Mitochondrial Loss of Hydrogen and Electric Gradient Under High Pressure

A high pressure of about 3 atmospheres was applied to an organ slice culture of rat liver. The rotating tubes that held the slice containers and culture media were sealed tightly to prevent gas leakage and connected to a gas supply. Pressurized oxygen were ventilated at intervals of 2.5 minutes. The mitochondria were treated with MitoTracker Red to identify whether they were permeable. Mitochondrial staining dyes were developed to study the morphology as well as the function of mitochondria. MitoTracker® Red is known to permeate passively into the plasma membrane and stay within active mitochondria along an electrical gradient (Poot M, et al. J Histochem Cytochem 1996;44:1363-1372). The cells were tested at the beginning of the culture at time zero and again after 3 hours. The results in Table 1 show that the mitochondrial staining was lost completely. This suggests that, under high pressure, the channel between mitochondria and the extracellular space opened and eventually lost its hydrogen gradient, resulting in failure of the mitochondria to produce ATP.

TABLE 1
confocal microscopy of mitochondria in pressure treated cells

| | |
|---|---|
| At the beginning of culture (time 0) | Confocal microscopy visualized |
| | mitochondria very well. |
| 3 hours after incubation with virus | Confocal microscopy did not |
| | visualize mitochondria at all. |
| | Mitochondria were not visible |

### Example 2

### Transfer of Genetic Material to Mitochondria Under Pressure

In order to evaluate the efficiency of gene transfer to mitochondria under high pressure, genetic materials were modified with respect to the mitochondrial genetic system by point mutagenesis. That is, the nucleic acid sequence was modified so that expression from the mitochondria translation system resulted in the same protein with same amino acid sequence as would be produced by cytoplasmic translation. The mitochondrial translation system differs by the presence of altered codon usage. For example, the UGA codon is a stop codon for protein synthesis using the universal code. However, in the mitochondrion, the UGA codes for tryptophan and the codons AGA and AGG code for arginine in the universal system but are stop codons in mitochondria.

The transfer of the genetic material did not require any chemicals that were necessary for conventional transfection through the plasma membrane, but only a pressure of 2-3 atmospheres was applied at intervals of 2.5 minutes for less than 3 hours.

The genetic material used was one of the most frequently used reporter genes, green fluorescent protein (GFP) (Zolotukhin S, et al. J Virol 1996;70:4646-4654). GFP was used to analyze the efficiency of gene transfer by modifying the nucleotide sequence of GFP as follows: Fifty-seven TGG codons of GFP were modified into TGA. TGA is a stop codon in the universal genetic code and tryptophan in the mitochondrial genetic code. The plasmid with the altered GFP was called GFP57. This plasmid can be used to identify where the protein is expressed, because if the GFP is translated within mitochondria, the full-length protein will be produced, while, in the cytoplasm (or any other part of the cell using the universal code), a truncated protein will be produced. GFP is particularly advantageous for this use because the full-length or wild-type protein will show up as green when the cells are viewed by fluorescent microscopy, while any truncated GFP will not show up at all.

The constructs produced are summarized in Table 2. For example, AGA codons which are known as stop codon in the mitochondrial genetic code were modified into CGT serially at the 73^{rd}, 96^{th}, 168^{th}, and 215^{th} codons of GFP57. In contrast, GFP73 was modified to have a CGT at the 73^{rd} codon, and GFP96 was modified to contain the CGT at the 73^{rd} and 96^{th} Other constructs are summarized in Table 2.

TABLE 2
GFP codons which were modified

| | | | | | | | | Mito- |
|---|---|---|---|---|---|---|---|---|
| | | | | | | | Conven- | chon- |
| | 57W | 73R | 96R | 168R | 215R | 239stop | tional | drial |
| | TGG | AGA | AGA | AGA | AGA | TAA | Tf | Tf |
| GFP | | | | | | | 238 | 72 |
| GFP57 | TGA | | | | | | 56 | 72 |
| GFP73 | TGA | CGT | | | | | 56 | 95 |
| GFP96 | TGA | CGT | CGT | | | | 56 | 167 |
| GFP168 | TGA | CGT | CGT | CGT | | | 56 | 214 |
| GFP215 | TGA | CGT | CGT | CGT | CGT | | 56 | 238 |

(Tf = transfection)

The plasmids were transfected into 293 cells using conventional transfection with calcium phosphate precipitation under ordinary conditions with and without pressure for comparison.

Two plasmids, GFP and GFP57 were transfected using the conventional calcium phosphate precipitation method and the presence of GFP analyzed using fluorescent microscopy. The control GFP showed a green fluorescent color under fluoromicroscopy and a full length GFP protein was identified using a western blot with commercial rabbit anti-GFP polyclonal antibody. GFP57 did not show any fluorescence because the stop codon is located in front prior to the fluorescent center at codon 65. In addition, the GFP57 was identified as a truncated protein using a western blot.

After transfection using the method above, the size and presence of fluorescence was used to determine whether the plasmids were delivered to the mitochondria as follows: the GFP will show fluorescence and a truncated protein at the 73^{rd} codon, GFP57 will be truncated at amino acid 73, GFP73 will be truncated at amino acid 96, GFP96 will be truncated at amino acid 168, GFP 168 will be truncated at amino acid 215 and GFP215 will have a full length GFP.

### Example 3

### Identification of the Optimum Pressure and Oxygen Concentration for Mitochondrial Gene Transfer

A knob was installed to the incubator to test adequate pressure for maximum gene transfer efficiency. The cells were incubated at various pressures (from 1.6 to 3.0 atmospheres) to identify which pressure or which pressure range was most conducive to the gene transfer of virus to mitochondria. The gene transfer was tested as above using the GFP and modified GFP constructs in Example 2.

According to a recent recommendation (Allen C B and White C W. Am J Physiol 1998;274:L159-L164) an oxygen concentration lower than 75% and preferably less than 50% oxygen concentration was tested for gene transfer. It was of interest to determine which oxygen concentration or range was most conducive to the delivery of virus to the mitochondria. Thus, a range of different oxygen concentrations from 50% to 95% were applied and the GFP gene transfer to mitochondria was analyzed to determine the oxygen concentration at which one gets the maximum efficiency.

### Example 4

### Establishment of Stable Transfection Cell Line

The method is used to establish a stable cell line containing HBV within the mitochondrion. LMH cells were grown and incubated with duck hepatitis B virus at 2-3 atmosphere for 3 hours. A series of cultures are tested to identify the presence of cccDNA (covalently closed circular DNA) within the mitochondria. Cells containing HBV cccDNA are grown and serially cultured. The cells are tested continuously over further generations to identify the continued presence of cccDNA within the mitochondria.

### Example 5

### Viral Growth in a Non-Natural Host

Immunodeficient nude mice are incubated within a hyperbaric oxygen chamber for varying hours after injection with human hepatitis B virus into the tail vein. Time calibration is important because prolonged exposure to high pressure may be hazardous for nude mice by causing caisson disease (compressed air sickness). At each timepoint, a serum and liver biopsy are tested for the presence of virus in the mitochondria by identifying the presence of HBV cccDNA within the mitochondria or alternatively, by identifying the presence of mitochondrially-expressed HBV proteins.

### Example 6

### Establishment of Transgenic Animal

Previously, a conventional method was used to produce HBV transgenic mice. The method, using needle puncture to establish transgenic mice, failed to show the production of viral cccDNA at all (Guidotti LG, et al. J Virol 1995;69:6158-6169). It is likely that the conventional methods did not allow for the entry of the virus or viral nucleic acid into the mitochondria. Thus, method disclosed herein is used for the production of transgenic mice containing viral DNA within the mitochondria.

Fertilized ova of mice are produced as follows: briefly, fertilized ova are incubated with hepatitis B virus under high pressure (2 atmospheres for 3 hours). The ova are used to produce transgenic mice according to conventional prior art methods. The transgenic mice are tested for the presence of cccDNA within the mitochondria.

### Example 7

### Transfer of Genetic Material to Lymphocytes or Dendritic Cells

The serum of Hepatitis C patients is incubated with mouse lymphocytes and/or dendritic cells under a pressure of 2.5 atmospheres. The viral titer is measured by standard commercial RT-PCR and the production of virus and viral antigens in the mitochondria is confirmed within one week. After confirmation, the immunogenic effects are evaluated in these cells.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods may be performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Similarly, the various features and steps discussed above, as well as other known equivalents for each such feature or step, can be mixed and matched by one of ordinary skill in this art to perform methods in accordance with principles described herein.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein, but instead by reference to claims attached hereto.

What is claimed is:

1. A method for the introduction of nucleic acid into an organelle of a host cell and/or tissue, comprising:
contacting the host cell and/or tissue with the nucleic acid at a pressure of between about 1.0 and about 3.0 atmospheres for a time sufficient to introduce the nucleic acid into the organelle.

2. The method of claim 1 further comprising applying an oxygenation of between about 50% and 95%.

3. The method of claim 2 wherein said oxygenation is for a time of less than about 3 hours.

4. The method of claim 1 further comprising testing the cells for the presence of the nucleic acid within the organelle.

5. The method of claim 1 wherein the nucleic acid is transferred into the organelle through a direct route from the extracellular space without entering the cytoplasm.

6. The method of claim 1, wherein the organelle is a mitochondrion.

7. The method of claim 1, wherein the organelle is a chloroplast.

8. The method of claim 1, wherein the pressure is between about 1.6 and 2.5 atmospheres.

9. The method of claim 1, wherein the pressure is between about 1.6 and about 1.8 atmospheres.

10. The method of claim 1, wherein the oxygenation is between about 75% and 95%.

11. The method of claim 1, wherein the oxygenation is about 95%.

12. The method of claim 1, wherein the cell is a human tissue culture cell.

13. The method of claim 1, wherein the nucleic acid is viral nucleic acid.

14. The method of claim 1, wherein the viral nucleic acid is carried by the virus.

15. The method of claim 1, wherein the nucleic acid is a vector.

16. The method of claim 1, further comprising contacting the cell with a protein or inorganic substance to enhance transfer of the nucleic acid.

17. The method of claim 13, wherein said protein is a viral protein.

18. The method of claim 1, wherein the nucleic acid is selected from the group consisting of: viral nucleic acid, recombinant DNA, recombinant RNA, and mixtures thereof.

19. The method of claim 1, wherein the organelle is a mitochondrion and the nucleic acid is translated according to the mitochondrial genetic code.

20. The method of claim 1 wherein the host cell is a non-natural host tissue or non-natural host cell.

21. The method of claim 1 wherein the nucleic acid is modified by point mutagenesis to match the mitochondrial genetic code.
